(19) United States
(12) Patent Application Publication
(10) Pub. No.: US 2008/0009505 A1
(43) Pub. Date: Jan. 10, 2008

(54) Theanine derivatives, uses thereof and processes for the manufacture thereof

(75) Inventors: George Hodges, Sharnbrook (GB); Jacek Paul Obuchowicz, Sharnbrook (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco Inc. d/b/a UNILEVER

(21) Appl. No.: 11/825,035

(22) Filed: Jul. 3, 2007

(30) Foreign Application Priority Data
Jul. 4, 2006 (EP) EP06116576

(51) Int. Cl. A61K 31/4965; A23F 3/00; A61P 25/04; C07D 241/04

(52) U.S. Cl. 514/255.01; 426/597; 544/383

(57) ABSTRACT

The present invention provides compounds, including peptides, which comprise theanine and/or theanine-like moieties. These compounds may deliver the physiological effects of theanine and/or offer enhanced storage stability. Also provided is a process for manufacturing the compounds by reacting theanine or a derivative thereof with at least one amino acid or a derivative thereof.

### TECHNICAL FIELD OF THE INVENTION

The present invention relates to derivatives of the amino acid theanine. The present invention also relates to the use of such compounds in pharmaceutical, cosmetic and food compositions as well as to processes for their manufacture.

### BACKGROUND OF THE INVENTION

Food and beverage products which can enhance the mental and physical aspects of the human body are becoming increasingly popular. In particular, products which produce an enhanced state of mental acuity are especially commercially valuable.

Theanine is an amino acid which, within the plant kingdom, is uniquely found in tea (Camellia sinensis). Theanine has been found to have numerous beneficial effects on the human body and mind. For example, it is reported that theanine stimulates α-waves in the mammalian brain and bestows a relaxed but alert feeling to the individual. These physiological effects are particularly apparent at specific dosage levels. For example, International patent application published as WO 2006/061097 (Unilever PLC et al.) describes consumable compositions which comprise specific levels of theanine and caffeine and which are shown to provide noticeable improvements in concentration, mental focus and/or alertness of an individual consuming the compositions. European patent application published as EP 1 393 726 A (Taiyo Kagaku KK) also discloses compositions for improving mental concentration containing theanine.

Although tea is relatively rich in theanine, in fact theanine only comprises about 1% by weight of the extractable tea solids in tea plant material. Thus, synthetic theanine (e.g. Suntheanine™ produced by Tayio Kagaku) has been developed to meet the increasing demand for products with enhanced levels of theanine. Furthermore, theanine is unstable in aqueous solution and is easily hydrolysed, especially at low pH. Thus formulators are required to include very high levels of theanine in their products in order to ensure that they maintain efficacy over the whole product shelf life.

Thus we have recognised that there is a need for new molecules that may deliver the physiological effects of theanine and/or offer enhanced storage stability. We have found that this object may be met by providing compounds that comprise theanine and/or theanine-like moieties.

### DEFINITIONS

Amino Acid Side Chain

Amino acids accord to the general formula (B):

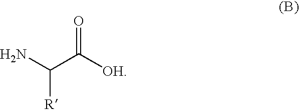

As used herein, the term “sidechain of an amino acid” refers to that part of the amino acid represented by the radical R′ in formula (B).

Theanine

Theanine is an amino acid having a glutamine side chain wherein the amide group on the side chain is ethyl-substituted. Thus theanine is also known as N-gamma-ethyl glutamine or 5-N-ethyl glutamine, and has the formula (A):

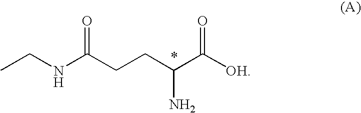

The naturally occurring form is L-theanine (N-gamma-ethyl-L-glutamine) wherein the chiral centre (i.e, the α-carbon denoted by * in formula (A)) has (S)-stereochemistry. However, unless specified otherwise the term theanine as used herein encompasses L-theanine, D-theanine (i.e., wherein the chiral centre has (R)-stereochemistry), and mixtures thereof L-theanine is preferred as this is believed to have the highest bio-activity.

By “theanine residue”, is meant a moiety according to formula (C):

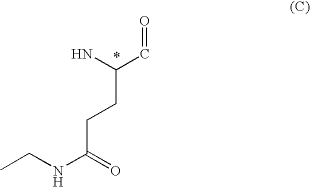

X₁, X₂ and X₃

The group X₁ is employed herein to represent a generic alkyl radical and is selected from linear, cyclic or branched alkyl and derivatives thereof. Such derivatives include alkyl with heteroatom such as hydroxyalkyl, alkyl with amine functionality and/or alkyl with carboxyl functionality. Preferred alkyls are C1-C8 alkyls. For example, the alkyl may be methyl, ethyl, propyl, isopropyl, butyl, t-butyl, pentyl, cyclopentyl, hexyl, cyclohexyl, heptyl or octyl.

The group X₂ is employed herein to represent a generic alkenyl radical and is selected from linear, cyclic or branched alkenyl and derivatives thereof. Such derivatives include alkenyl with heteroatom such as hydroxyalkenyl, alkenyl with amine functionality and/or alkenyl with carboxyl functionality. Preferred alkenyls are C2-C8 alkenyls. For example, the alkenyl may be ethenyl, propenyl, isopropenyl, butenyl, t-butenyl, pentenyl, cyclopentenyl, hexenyl, cyclohexenyl, heptenyl or octenyl. The term alkenyl also encompasses dienyl and higher levels of unsaturation.

The group X₃ is employed herein to represent a generic aryl radical and is selected from aromatic radicals and derivatives thereof. Such derivatives include aryl with heteroatom such as hydroxyaryl, aryl with amine functionality and/or aryl with carboxyl functionality. Preferred aryls are C6-C12 aryls. For example, the aryl may be phenyl, alkylphenyl, benzyl, alkylbenzyl, or naphthyl.

Peptide

The term “peptide” as used herein means a compound comprising two or more amino acid residues joined by at least one peptide bond.

### SUMMARY OF THE INVENTION

In a first aspect, the present invention provides compounds of general formula (1): 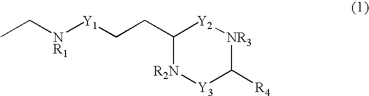
wherein

R₁, R₂, and R₃ are each independently selected from the group consisting of H, C(O)X and C(O)OX, wherein for each of R₁, R₂ and R₃, X is independently selected from the group consisting of H, X₁, X₂ and X₃;

R₄ is the side chain of an amino acid or a derivative thereof; and

Y₁, Y₂ and Y₃ are each independently selected from the group consisting of C═O and 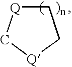
wherein Q and Q′ are each independently selected from the group consisting of O, N, and S, and n is 1 or 2.

Compounds of general formula (1) may deliver the physiological effects of theanine and/or offer enhanced storage stability.

In a further aspect, the present invention provides a compound which is a peptide comprising at least two theanine residues.

Such peptides may deliver the physiological effects of theanine and/or offer enhanced storage stability.

In a yet further aspect, the present invention provides compositions comprising the compounds of the invention. These compositions are preferably suitable for administering the compound to an individual either as a pharmaceutical, cosmetic or foodstuff.

In a still further aspect, the invention provides use of the compounds or compositions of the invention as a medicament or in the preparation of a medicament. In particular, the compounds of the present invention may be administered to an individual to provide a noticeable improvement in concentration, mental focus and/or alertness; and/or for giving a noticeable improvement in sleep quality; and/or for giving a noticeable improvement in creativity; and/or for maintaining or improving the performance of an immune system; and/or for improving digestion; and/or for managing bodyweight; and/or for reducing mental stress; and/or for treating behavioural disorders; and/or for treating premenstrual syndrome; and/or for treating headache and/or migraine.

In a yet another aspect, the present invention provides a process for manufacturing the compounds of the invention. The process comprising the step of reacting theanine or a derivative thereof with at least one amino acid or a derivative thereof. This process allows for a relatively simple route to the compounds of the invention and utilises readily available raw materials, i.e., amino acids and their derivatives.

### DETAILED DESCRIPTION

Compounds

The present invention provides compounds of general formula (1):

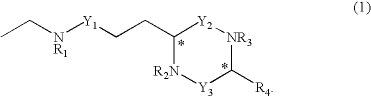

These compounds contain a theanine residue, although the residue may be substituted with labile groups that are easily converted in-vivo to yield the theanine residue structure. Thus R₁ and R₂ are each independently selected from the group consisting of H, C(O)X and C(O)OX, wherein for each of R₁ and R₂, X is independently selected from the group consisting of H, X₁, X₂ and X₃; and Y₁ and Y₂ are each independently selected from the group consisting of C═O and 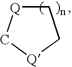
wherein Q and Q′ are each independently selected from the group consisting of O, N, and S, and n is 1 or 2.

The present invention also encompasses compounds which may re-arrange in-vivo to yield a compound of formula (1). For example, the amide group of the theanine side chain may be formed by the Beckmann rearrangement of an oxime group.

Formula (1) should also be understood to encompass salt forms of any functional groups therein.

In a preferred embodiment, the theanine residue is not substituted and the compound has formula (2):

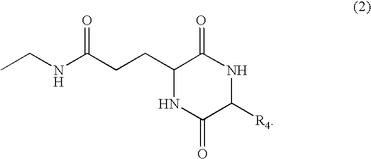

The theanine residue of the compounds of formula (1) and/or (2) is coupled through a cyclic structure to a second amino acid residue such that R₄ is the side chain of an amino acid or a derivative thereof. Preferably, R₄ is the side chain of an amino acid selected the group consisting of theanine, glycine, alanine, valine, leucine, isoleucine, glutamine, lysine, hydroxylysine, histidine, arginine, phenylalanine, asparagine, tyrosine, tryptophan, thyroxine, serine, threonine, cysteine, methionine, norvaline, ornithine, aspartic acid, glutamic acid, and derivatives thereof.

Suitable derivatives of the amino acid are preferably those wherein the amino acid side chain is substituted with labile groups that are easily converted in-vivo to yield the amino acid. In particular, any amine groups on the amino acid side chain may be substituted with a functionality selected from the group consisting of C(O)X and C(O)OX, wherein X is selected from the group consisting of H, X₁, X₂ and X₃. Alternatively or additionally, any carbonyl groups on the amino acid side chain may be replaced by 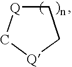
wherein Q and Q′ are each independently selected from the group consisting of O, N, and S, and n is 1 or 2.

Similarly, the backbone of the second amino acid residue may also be substituted with labile groups. Thus R₃ is selected from the group consisting of H, C(O)X and C(O)OX, wherein X is selected from the group consisting of H, X₁, X₂ and X₃; and Y₃ is selected from the group consisting of C═O and 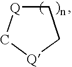
wherein Q and Q′ are each independently selected from the group consisting of O, N, and S, and n is 1 or 2.

In a preferred embodiment, the second amino acid residue is also a theanine residue or a derivative thereof such that R₄ is the side chain of theanine or a derivative thereof and has the formula (3): 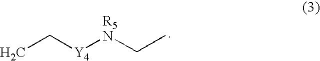
wherein R₅ is selected from the group consisting of H, C(O)X and. C(O)OX, wherein X is X₁, X₂ or X₃; and Y₄ is selected from the group consisting of C═O and 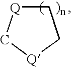
wherein Q and Q′ are each independently selected from the group consisting of O, N, and S, and n is 1 or 2.

In a preferred embodiment, in order to maximise the theanine-like properties of the compound, it is a dimer of theanine and has the formula (4):

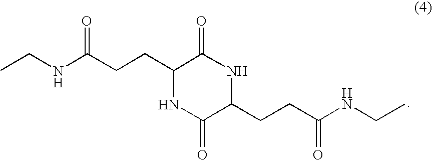

In the compounds of formulae (1) to (4), the chiral centres (denoted * in formula (1) above) may be (S), (R) or a combination thereof. Preferably, however, both chiral centres have (S)-stereochemistry.

The present invention also provides a compound which is a peptide comprising at least two theanine residues. Such peptides may deliver the physiological effects of theanine and/or offer enhanced storage stability.

To maximise the theanine-like properties of the peptide, it is preferred that the compound comprises at least 50% theanine residues, more preferably at least 75% theanine residues and most preferably from 90 to 100% theanine residues by weight of the compound. In a preferred embodiment the peptide is a dimer of theanine, most preferably a dimer according to formula (4).

Preferably also, at least 50% of the theanine residues present in the peptide are residues of L-theanine (i.e. retain the stereochemistry of L-theanine about the chiral centre marked * in formula (C)), more preferably at least 75% and most preferably from 95 to 100%.

Compositions

The compounds of the present invention may be employed in substantially pure form or may comprise part of a composition. These compositions are preferably suitable for administering the compound to an individual, e.g. in the form of a pharmaceutical or cosmetic composition. The preferred mode of administration is oral and so it is preferred that the composition is edible, most preferably as a foodstuff such as a beverage.

The effective amount of the compound of the invention which is included in a composition will depend on the exact nature of the composition and the intended benefit delivered by the composition. Typically, however, the composition comprises the compound in an amount from 0.000001 to 50% by weight of the composition, preferably from 0.0001 to 20%, more preferably from 0.001 to 10% and most preferably from 0.01 to 5%.

The compositions preferably comprise other active components which can be derived from tea (although such active components need not actually be derived from tea). Thus the compositions preferably comprise a component selected from the group consisting of caffeine, catechins, theaflavins, thearubigens, theanine, GABA (gamma-aminobutyric acid) and mixtures thereof.

Pharmaceutical and Cosmetic Compositions

The pharmaceutical and cosmetic compositions of the present invention may be suitable for any form of administration including oral, topical and/or intravenous administration. The form of the composition may, among others, be a tablet, pill, lozenge, paste, lotion, gel, cream, liquid (including emulsion), spray (including aerosol spray), foam or powder.

The pharmaceutical or cosmetic composition comprises a pharmaceutically acceptable vehicle which may act as a diluent, dispersant or carrier for the inventive compounds in the composition. The vehicle may be aqueous or anhydrous.

Water, when present, will be in amounts which may range from 5 to 99%, preferably from 20 to 70%, optimally between 40 and 70% by weight of the composition.

Besides water, relatively volatile solvents may also be included within the vehicle. Most preferred are monohydric C1-C3 alkanols. These include ethyl alcohol, methyl alcohol and isopropyl alcohol.

Emollient materials may also be included in the vehicle. These may be in the form of silicone oils and/or synthetic esters.

Humectants of the polyhydric alcohol type may also be employed in the vehicle. Typical polyhydric alcohols include glycerol, polyalkylene glycols and more preferably alkylene polyols and their derivatives.

Thickeners may also be utilized as part of the vehicle, as may sugars, plasticizers, antioxidants, chelating agents, buffers, coloring agents, pigments, opacifiers, surfactants, propellants, flavours and/or perfumes.

Preferred cosmetic compositions are those suitable for application to human skin and preferably include a skin benefit agent in addition to the inventive compound. Suitable skin benefit agents include anti-aging, wrinkle-reducing, skin whitening, anti-acne, sunscreen and sebum reduction agents. Examples of these include alpha-hydroxy acids, beta-hydroxy acids, polyhydroxy acids, hydroquinone, t-butyl hydroquinone, vitamin B and C and their derivatives, micronised metal oxides, retinoids, betulinic acid, vanillic acid, allantoin, a placenta extract, hydrolactin, resorcinol derivatives, and mixtures thereof.

Food Compositions

A particularly preferred form of the composition is that of a foodstuff, as this allows for convenient and enjoyable consumption of the compounds of the invention. The food composition may be, for example, a margarine, low fat spread, confectionery product (such as chocolate), ice cream, dressing, mayonnaise, sauce, bakery product, shortening or cheese. However, it is especially preferred that the food composition is a beverage.

The food may be dried and contain less than 40% water by weight of the composition, preferably less than 25%, more preferably from 1 to 15%. Alternatively, the food may be substantially aqueous and contain at least 40% water by weight of the composition, preferably at least 50%, more preferably from 65 to 99.9%.

The food preferably comprises nutrients including carbohydrate, protein, fat, vitamins, minerals and mixtures thereof. The food may be low calorie (e.g. have an energy content of less than 100 kCal per 100 g of the composition) or may have a high calorie content (e.g. have an energy content of more than 100 kCal per 100 g of the composition; preferably between 150 and 1000 kcal).

The food may also contain salt, flavours, colours, preservatives, antioxidants and mixtures thereof.

When the food composition is a beverage, it will typically comprise at least 85% water, more preferably at least 90%, optimally between 95 and 99.9% by weight of the beverage. Preferably the beverage is a coffee-based beverage, a tea-based beverage and/or a cocoa-based beverage.

The pH of the beverage may, for example, be from 2.5 to 8, preferably 3 to 6, more preferably from 3.5 to 5.

Most preferably the beverage is tea and comprises tea-derived solids such as catechins, theaflavins, thearubigens and mixtures thereof. The tea solids may, for example, comprise from 0.005 to 3% by weight of the beverage, more preferably from 0.01 to 2%, most preferably from 0.05 to 1% by weight of the beverage.

Use of the Compounds or Compositions

The compounds and compositions of the present invention may be used to deliver any of the benefits associated with theanine. In particular, the compounds and/or compositions may be used as a medicament or in the manufacture of a medicament for providing at least one of the following benefits:
improving concentration, mental focus and/or alertness;
improving sleep quality;
improving creativity;
improving the immune system;
improving digestion;
managing bodyweight;
reducing mental stress;
treating behavioural disorders;
treating premenstrual syndrome;
treating headache and/or migraine.

For example, the compound or composition may be used in a method of providing at least one of these benefits to an individual, the method comprising administering to the individual the compound or composition. Preferably the compound and/or composition is administered orally.

Manufacture of the Compounds

The compounds of the present invention may be manufactured by any suitable process. However, a preferred process for manufacturing the compound comprises the step of reacting theanine or a derivative thereof with at least one amino acid or a derivative thereof.

Suitably, the reaction involves coupling of the amino-acid backbones of the theanine and the at least one amino acid. Thus it is preferred that the reaction comprises forming at least one peptide bond between the theanine or derivative thereof and the at least one amino acid or derivative thereof. More preferably, the reaction comprises forming two peptide bonds between the theanine or derivative thereof and the at least one amino acid or derivative thereof. The peptide bonds may, for example, be formed as a result of nucleophilic attack at the terminal carbonyl group of theanine by the amino group of the amino acid and nucleophilic attack at the carbonyl of the amino acid by the amino group of theanine.

The formation of the peptide bonds may, for example, be achieved by heating a mixture of theanine and amino acid to high temperature (170 to 200° C.) in an organic solvent with a silica catalyst, as described in V. A. Basiuk et al., Synthesis-Stuttgart, 1992, 5, pp. 449-451. However, in order to avoid such harsh reaction conditions it is preferred that the terminal carbonyl of the theanine and/or the amino acid is activated. Suitable activation means are well-known to those in the art and usually involve replacing the OH group of the carboxylic acid functionality with a group such as acyl chloride, anhydride, ester and the like. Thus the theanine or derivative thereof for use in the process is preferably according to formula (6): 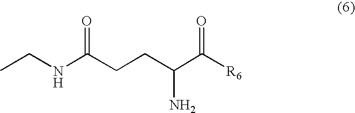
wherein R₆ is selected from the group consisting of Cl, OC(O)X, OX, OC(NX)NHX′, OC(CH₂)OX, OC(O)OX, OP(O)Ph₂, OP⁺(X) (X′)X″, OP(O) (OX)OX′, N(X)N(X′)X″, and N₃ and wherein each of X, X′ and X″ is independently selected from the group consisting of H, X₁, X₂ or X₃.

For preparing compounds of formula (1), the process preferably comprises the reaction shown in scheme (I): 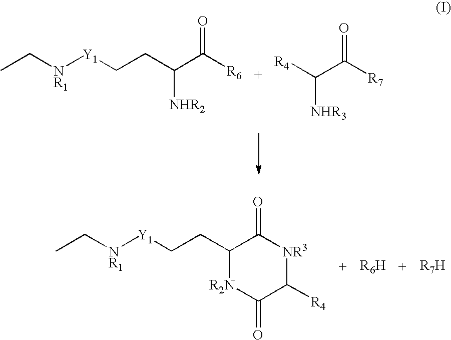
wherein R₇ is selected from the group consisting of Cl, OC(O)X, OX, OC(NX)NHX′, OC(CH₂)OX, OC(O)OX, OP(O)Ph₂, OP⁺(X)(X′)X″, OP(O)(OX)OX′, N(X)N(X′)X″, and N₃ and wherein each of X, X′ and X″ is independently selected from the group consisting of H, X₁, X₂ or X₃.

Nucleophilic substitution to form peptide bonds is promoted at high pH, therefore it is preferred that the reaction of the theanine or derivative thereof with the at least one amino acid or derivative thereof occurs at a pH of from 8 to 14, more preferably 10 to 12.

### EXAMPLES

### Example 1

This example demonstrates the manufacture of a compound of the invention (N-ethyl-3-[5-(2-ethylcarbamoyl-ethyl)-3,6-dioxo-piperazin-2-yl]-propionamide; see formula (4)).

L-Theanine (5 g) (Suntheanine™) was slurried in dry MeOH (50 ml). Then 5.145 g of Cl₂SO were added drop-wise over 5 minutes (part way through the addition all of the solids dissolve to give a clear colourless solution) and the resulting solution was held for 12 hours at 20° C. to allow the reaction shown in scheme (II) to occur:

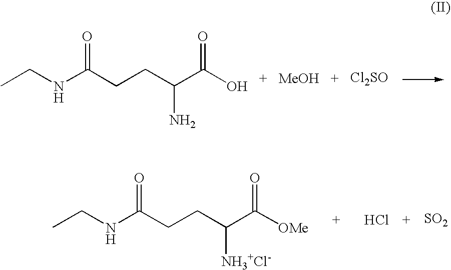

MeOH, SO₂ and HCl were then removed under vacuum to leave a thick clear oil to which dry MeOH (50 ml) was added. The pH was then raised by adding MeONa while testing pH by intermittently spotting onto wet indicator paper. After adding 3.17 g of MeONa, the pH had increased to >11, which resulted in the reaction shown in scheme (III):

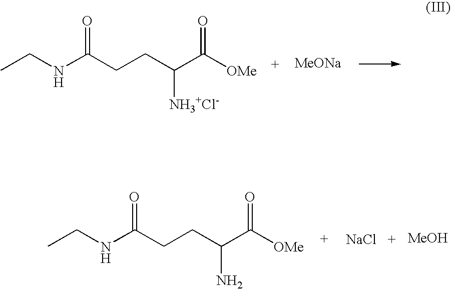

The resulting solution was held for 12 hours at 20° C. to allow the reaction shown in scheme (IV) to occur:

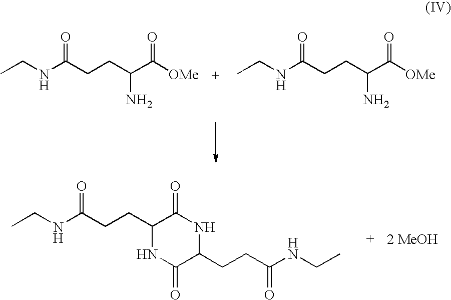

The resulting suspension was then filtered to remove NaCl and the filtrate subjected to vacuum to remove MeOH. The product was a white solid.

The product was analysed with LC-MS (Liquid-Chromatography coupled Mass Spectroscopy) under the following conditions:
Column: Phenomonex Gemini 5μ C18 110A 150 mm ×2.00 mm
Solvents: A: 0.1% Formic Acid/Water B: 0.1% Formic Acid/Acetonitrile
Flow rate: 0.2 ml/minute
Column Temperature: 30° C.

Stop Time: 25 minutes

Solvent Gradient:

| Time (minutes) | % A | % B |
|---|---|---|
| 0 | 80 | 20 |
| 10 | 99 | 1 |
| 11 | 0 | 100 |
| 14 | 0 | 100 |
| 15 | 99 | 1 |

Mass Spectrometer Detection:
   Selected Ion Recording at m/z 175.6 for Theanine.
   Selected Ion Recording at m/z 313.5 for Theanine dimer.

The results showed that the product was free from theanine (retention time of theanine=3.7 min) and had a characteristic new peak with a retention time of around 7.4 min.

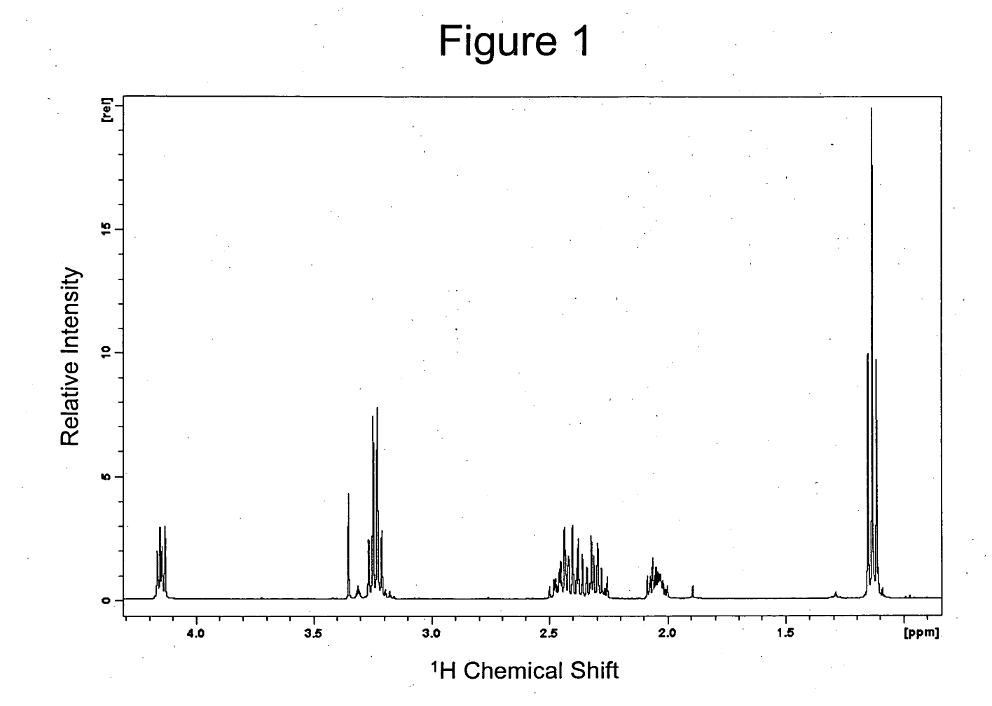

The high resolution proton NMR spectrum of the product in MeOD-d₄ is shown in FIG. 1.

### Example 2

This example demonstrates the superior stability of the theanine dimer (N-ethyl-3-[5-(2-ethylcarbamoyl-ethyl)-3,6-dioxo-piperazin-2-yl]-propionamide) prepared in Example 1 compared with that of theanine.

0.05 M citrate buffer with a pH of 4.0 at 20.3° C. was prepared. To this buffer were added 5.35 mM theanine and 2.675 mM of the theanine dimer. The resulting solution was held at 92° C. for two weeks with small samples being taken regularly for analysis with HPLC. The results are shown in Table 1:

TABLE 1

| Storage Time | Theanine Remaining | Theanine Dimer Remaining |
|---|---|---|
| (hours) | (mM) | (mM) |
| 0.0 | 5.35 | 2.68 |
| 2.5 | 4.83 | 2.75 |
| 17.5 | 2.54 | 2.80 |
| 26.3 | 1.83 | 2.78 |
| 42.0 | 0.81 | 2.99 |
| 50.0 | 0.05 | 3.31 |
| 121.3 | 0.04 | 3.36 |
| 137.3 | 0.03 | 3.38 |
| 145.8 | 0.02 | 3.51 |
| 161.6 | 0.01 | 3.54 |
| 170.2 | 0.01 | 3.59 |
| 185.8 | 0.01 | 3.63 |
| 193.3 | 0.01 | 3.80 |

The slight increase in the concentration of theanine dimer with time was due to gradual evaporation of water from the container which could not be completely sealed during the experiment. However, it is clear from the data in Table 1 that the theanine dimer is much more stable than theanine.

1. A compound of formula (1): 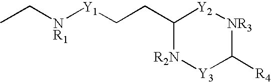
(1) wherein
R₁, R₂, and R₃ are each independently selected from the group consisting of H, C(O)X and C(O)OX, wherein for each of R₁, R₂, and R₃, X is independently selected from the group consisting of
H,
linear, cyclic or branched alkyl and derivatives thereof,
linear, cyclic or branched alkenyl and derivatives thereof, and
aromatic radicals and derivatives thereof;
R₄ is the side chain of an amino acid or a derivative thereof; and
Y₁, Y₂ and Y₃ are each independently selected from the group
consisting of C═O and 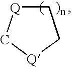
wherein Q and Q′ are each
independently selected from the group consisting of O, N, and S, and n is 1 or 2.

2. A compound according to claim 1 wherein Y₁, Y₂, and Y₃ are C═O.

3. A compound according to claim 1 wherein R₁, R₂ and R₃ are H.

4. A compound according to claim 1 wherein R₄ is the side chain of an amino acid selected from the group consisting of theanine, glycine, alanine, valine, leucine, isoleucine, glutamine, lysine, hydroxylysine, histidine, arginine, phenylalanine, asparagine, tyrosine, tryptophan, thyroxine, serine, threonine, cysteine, methionine, norvaline, ornithine, aspartic acid, glutamic acid, and derivatives thereof.

5. A compound according to claim 4 wherein R₄ is the side chain of theanine or a derivative thereof.

6. A compound according to claim 5 wherein R₄ is the side chain of theanine.

7. A compound according to claim 6 which is a dimer of theanine.

8. A compound which is a peptide comprising at least two theanine residues.

9. A compound according to claim 8 comprising at least 50% by weight of theanine residues, more preferably at least 75%.

10. A pharmaceutical or cosmetic composition comprising a compound according to claim 1 and a pharmaceutically acceptable carrier.

11. A food composition comprising a compound according to claim 1.

12. A food composition according to claim 11 which is a beverage, preferably a tea-based beverage.

13. A composition according to claim 10 wherein the compound is present in an amount from 0.000001 to 50% by weight of the composition.

14. Use of a compound according to claim 1 for giving a noticeable improvement in concentration, mental focus and/or alertness; and/or for giving a noticeable improvement in sleep quality; and/or for giving a noticeable improvement in creativity; and/or for maintaining or improving the performance of an immune system; and/or for improving digestion; and/or for managing bodyweight; and/or for reducing mental stress; and/or for treating behavioural disorders; and/or for treating premenstrual syndrome; and/or for treating headache and/or migraine.

15. A method of:
improving the concentration, mental focus and/or alertness of an individual; and/or
improving the sleep quality of an individual; and/or
improving the creativity of an individual; and/or
improving the immune system of an individual; and/or
improving the digestion of an individual; and/or
managing the bodyweight of an individual; and/or
reducing the mental stress of an individual; and/or
treating a behavioural disorder of an individual; and/or
treating premenstrual syndrome of an individual; and/or
treating headache and/or migraine of an individual;
the method comprising administering to the individual a compound according to claim 1.

16. A process for manufacturing a compound according to claim 1, the process comprising the step of reacting theanine or a derivative thereof with at least one amino acid or a derivative thereof.

17. A process according to claim 16 wherein the theanine or derivative thereof has the formula (6): 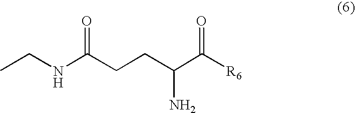
wherein R6 is selected from the group consisting of Cl, OC(O)X, OX, OC(NX)NHX′, OC(CH₂)OX, OC(O)OX, OP(O)Ph₂, OP⁺(X)(X′)X″, OP(O)(OX)OX′, N(X)N(X′)X″, and N₃ and wherein each of X, X′ and X″ is independently selected from the group consisting of H,
linear, cyclic or branched alkyl and derivatives thereof,
linear, cyclic or branched alkenyl and derivatives thereof, and
aromatic radicals and derivatives thereof.

18. A process according to claim 16 wherein the at least one amino acid or derivative thereof is selected from the group consisting of theanine, glycine, alanine, valine, leucine, isoleucine, glutamine, lysine, hydroxylysine, histidine, arginine, phenylalanine, asparagine, tyrosine, tryptophan, thyroxine, serine, threonine, cysteine, methionine, norvaline, ornithine, aspartic acid, glutamic acid, derivatives thereof and mixtures thereof.

19. A process according to claim 18 wherein the amino acid or derivative thereof is theanine or a derivative of theanine.
